(19) United States
(12) Patent Application Publication
(10) Pub. No.: US 2016/0107992 A1
(43) Pub. Date: Apr. 21, 2016

(54) INTERMEDIATES USEFUL FOR THE PREPARATION OF SAXAGLIPTIN

(71) Applicant: CHEMELECTIVA S.R.L, Novara (IT)

(72) Inventors: Graziano CASTALDI, Novara (IT); Giuseppe PALLANZA, Novara (IT); Mauro GABOARDI, Novara (IT); Marta CASTALDI, Novara (IT)

(21) Appl. No.: 14/894,770

(22) PCT Filed: Jun. 18, 2014
(86) PCT No.: PCT/EP2014/062825

§ 371 Date: Nov. 30, 2015

(30) Foreign Application Priority Data
Jun. 20, 2013 (IT) MI2013A001029

(51) Int. Cl. C07D 209/52; C07D 207/22

(52) U.S. Cl. CPC C07D 209/52; C07D 207/22

(57) ABSTRACT

Intermediates useful for the synthesis of saxagliptin and their preparationare described.

The present invention relates to intermediates useful for the synthesis of saxagliptin and their preparation.

Saxagliptin is a selective, reversible and competitive inhibitor of dipeptidyl-peptidases 4 (DPP-4), enzymes that degrade the incretin hormones, which is used in adults suffering from diabetes mellitus type 2 (non-insulin dependent diabetes) to improve the control of blood glucose levels.

Incretins are hormones produced in the gastrointestinal region and they are mainly GLP-1 (Glucagon-Like Peptide 1) and GIP (Glucose-dependent Insulinotropic Peptide). They are secreted after meals, particularly GLP-1, and have the function of controlling glycemia in different ways: increase of insulin secretion by pancreatic beta cells, decrease of glucagon secretion (insulin antagonist) by pancreatic alpha cells, slowdown of motility and of gastric empty with the consequent decrease in appetite.

GLP-1 is rapidly degraded into an inactive peptide by DDP-4, moreover its production decreases when glycemia decreases, its control over the latter is then calibrated and “when needed” thus avoiding hypersecretion of insulin and consequent dangerous hypoglycemia.

In diabetic patients, the natural action of GLP-1 is defective, it was therefore thought to restore this activity for exploiting it, particularly for the oral therapy of diabetes mellitus type 2, a disorder in which pancreas is not able to produce enough insulin to control blood glucose levels or in which the body is not able to effectively use insulin with the consequent advantage of decreasing the various and problematic side effects due to a prolonged oral therapy with the traditional drugs.

Saxagliptin, acting as DPP-4 inhibitor, inhibits the degradation of incretin hormones in the body, particularly GLP-1, increasing their level in the blood and stimulating the pancreas to produce more insulin when there is a high glycemic level, thus decreasing the amount of glucose produced by the liver, it also decreases glucagone levels, allowing the control of diabetes mellitus type 2.

Saxagliptin is a compound of formula (I)

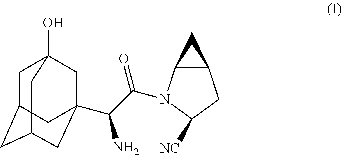 [SMILES: N#C[C@@H]1C[C@@H]2C[C@@H]2N1C(=O)[C@@H](N)C12CC3CC(CC(O)(C3)C1)C2]

chemically known as (1S,3S,5S)-2-[(2S)-2-amino-2-(3-hydroxy-1-adamantyl)acetyl]-2-azabicyclo[3.1.0]hexan-3-carbonitrile, disclosed in U.S. Pat. No. 6,395,767 and commercially sold under the name Onglyza®.

Various saxagliptin syntheses are known in the literature.

U.S. Pat. No. 6,395,767 discloses the synthesis of saxagliptin by condensation reaction between the cyclopropane derivative of L-proline of formula (II)

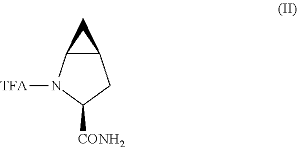

and the adamantane derivative of formula (III)

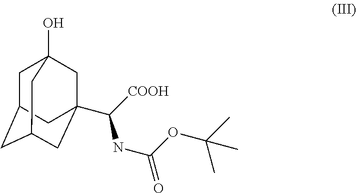 [SMILES: CC(C)(C)OC(=O)N[C@H](C(=O)O)C12CC3CC(CC(O)(C3)C1)C2]

to give the compound of formula (IV)

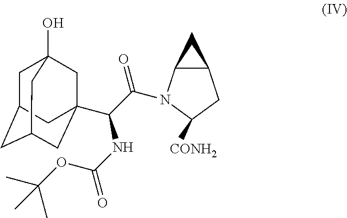 [SMILES: CC(C)(C)OC(=O)N[C@H](C(=O)N1[C@H](C(N)=O)C[C@@H]2C[C@@H]21)C12CC3CC(CC(O)(C3)C1)C2]

followed by the protection of the hydroxyl group, the reduction of the amide group and the deprotection of the amino and hydroxyl group. Processes for the preparation of the intermediates of formula (II) and (III) are furthermore disclosed.

U.S. Pat. No. 7,214,702 discloses a process for the synthesis of saxagliptin wherein the compound of formula (IV) is subjected to direct “one-pot” dehydration by treatment with phosphorus oxychloride in the presence of an organic solvent and subsequent treatment with water.

U.S. Pat. No. 7,705,033, U.S. Pat. No. 7,741,082 and WO 10/32129 disclose a process for the synthesis of saxagliptin by condensation reaction between the compound of formula (III) and a salt of the compound of formula (V)

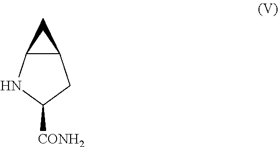

Moreover they disclose an enzymatic process for the preparation of the compound of formula (III).

U.S. Pat. No. 7,186,846 discloses a synthetic process wherein the compound of formula (III) is subjected to a coupling reaction with the compound of formula (V), through its protected derivative, and to the subsequent conversion into a chloro derivative by reaction with a Vilsmeier reagent or another chlorinated reactant.

U.S. Pat. No. 7,250,529 discloses a process for the synthesis of an intermediate useful for the synthesis of saxagliptin, 2-(3-hydroxy-1-adamantyl)-2-oxoacetic acid, by treatment of 1-acetyl-3-hydroxyadamantane with permanganate.

WO 06/128952 discloses the synthesis of 2-(3-hydroxy-1-adamantyl)-2-oxoacetic acid by treatment of an acyl derivative of adamantane with an oxidizing agent in the presence of a base.

The processes known in the art involve the use of reactants which are hard to handle and with a high environmental impact. Moreover, many processes described in the state of the art comprise several steps of protection and deprotection of the functional groups that make the entire process expensive.

We have now found new compounds that are intermediates useful for the synthesis of saxagliptin and can be synthesized without requiring several steps of protection and deprotection of the functional groups in an economically advantageous way with low environmental impact.

Object of the present invention are therefore the compounds of formula (VI)

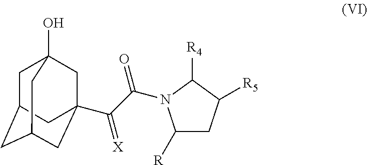

wherein X is an oxygen atom or a N—R₁ group;

R is a CONH₂ group, a COOR₂ group or a CN group;

R₁ is a hydrogen atom, a hydroxy group, an OR₃ group or an optionally substituted benzyl group;

R₂ is a linear or branched C₁₋₁₀ alkyl group, an optionally substituted benzyl group;

R₃ is a linear or branched C₁₋₁₀ alkyl group, an acetyl group, an optionally substituted benzyl group, a SO₂R₆ group;

R₄ and R₅ taken together form a double bond or a three member ring;

R₆ is a linear or branched C₁₋₁₀ alkyl or an optionally substituted group.

The compounds of formula (VI) are an object of the present invention in all their possible stereochemical configurations for all their stereogenic atoms.

Particularly, a preferred object of the present invention are the following compounds in all their possible stereochemical configurations for all their stereogenic atoms:

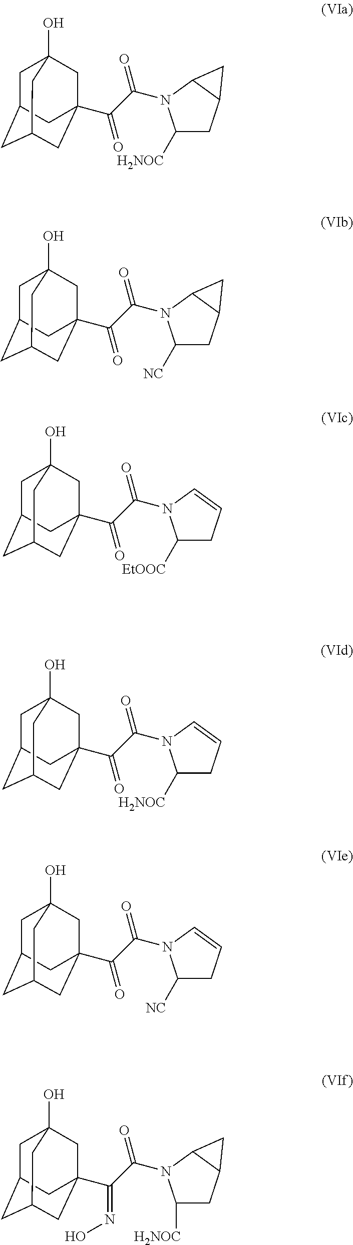 [SMILES: NC(=O)C1CC2CC2N1C(=O)C(=O)C12CC3CC(CC(O)(C3)C1)C2 ; N#CC1CC2CC2N1C(=O)C(=O)C12CC3CC(CC(O)(C3)C1)C2 ; CCOC(=O)C1CC=CN1C(=O)C(=O)C12CC3CC(CC(O)(C3)C1)C2 ; NC(=O)C1CC=CN1C(=O)C(=O)C12CC3CC(CC(O)(C3)C1)C2 ; N#CC1CC=CN1C(=O)C(=O)C12CC3CC(CC(O)(C3)C1)C2 ; NC(=O)C1CC2CC2N1C(=O)/C(=N/O)C12CC3CC(CC(O)(C3)C1)C2]

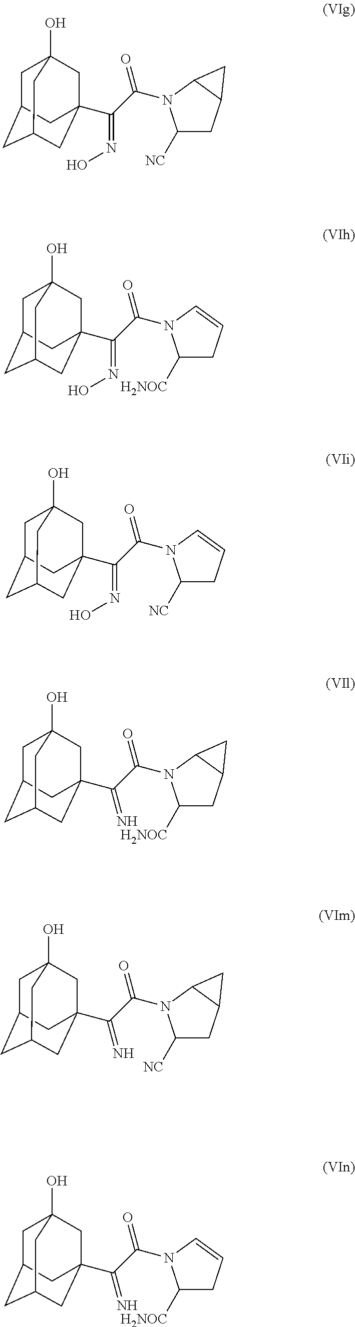 [SMILES: N#CC1CC2CC2N1C(=O)/C(=N/O)C12CC3CC(CC(O)(C3)C1)C2 ; NC(=O)C1CC=CN1C(=O)/C(=N/O)C12CC3CC(CC(O)(C3)C1)C2 ; N#CC1CC=CN1C(=O)/C(=N/O)C12CC3CC(CC(O)(C3)C1)C2 ; N=C(C(=O)N1C(C(N)=O)CC2CC21)C12CC3CC(CC(O)(C3)C1)C2 ; N#CC1CC2CC2N1C(=O)C(=N)C12CC3CC(CC(O)(C3)C1)C2 ; N=C(C(=O)N1C=CCC1C(N)=O)C12CC3CC(CC(O)(C3)C1)C2]

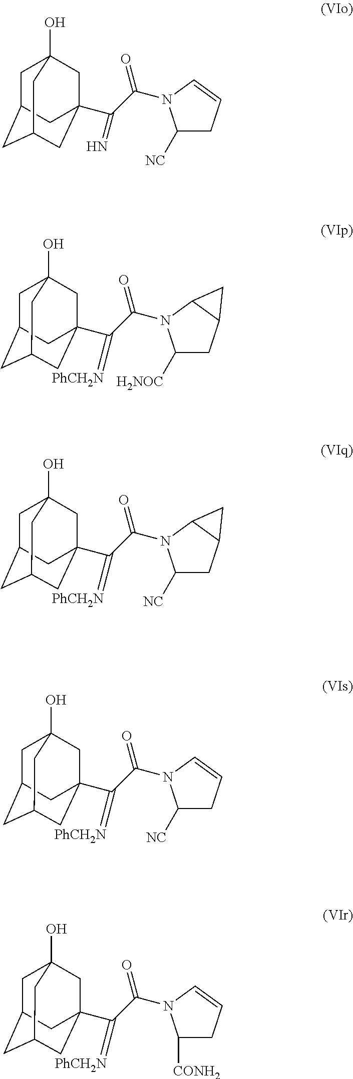

A further object of the present invention is a process for the synthesis of the compounds of formula (VI) comprising:
a) the condensation reaction between the adamantane derivative of formula (VII)

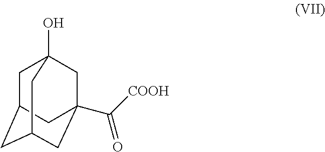 [SMILES: O=C(O)C(=O)C12CC3CC(CC(O)(C3)C1)C2]

and a compound of formula (VIII)

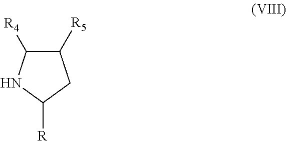

wherein
R is a CONH₂ group, a COOR₂ group or a CN group;
R₂ is a linear or branched C₁₋₁₀ alkyl or an optionally substituted benzyl group;
R₄ and R₅ taken together form a double bond or a three member ring;
in the presence of a condensing agent and optionally in the presence of a base in an aprotic solvent to give the compounds of formula (VI) wherein X is an oxygen atom;
the optional reaction of the compound of formula (VI) obtained in step a) with NH₂R₁ in the presence of a base or an acid in a polar solvent to give the compounds of formula (VI) wherein X is a N—R₁ group, wherein:
R₁ is a hydrogen atom, a hydroxy group, an OR₃ group or an optionally substituted benzyl group;
R₃ is a linear or branched C₁₋₁₀ alkyl group, an acetyl group, an optionally substituted benzyl group or a SO₂R₆ group;
R₆ is a linear or branched C₁₋₁₀ alkyl or an optionally substituted aryl group.

Alternatively, the compounds of formula (VI) can be prepared through a process which is a further object of the present invention comprising:
a1) the reaction of the adamantane derivative of formula (VII) with NH₂R₁ in the presence of a base or an acid, in a polar solvent to give the compounds of formula (IX)

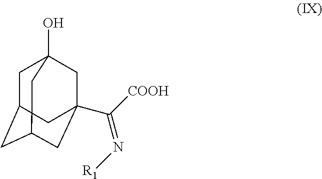

wherein R₁ is a hydrogen atom, a hydroxy group, an OR₃ group or an optionally substituted benzyl group;
R₃ is a linear or branched C₁₋₁₀ alkyl, an acetyl group, an optionally substituted benzyl group or a SO₂R₆ group;
R₆ is a linear or branched C₁₋₁₀ alkyl or an optionally substituted aryl group.
b1) the condensation reaction between the compound of formula (IX) and the compound of formula (VIII)

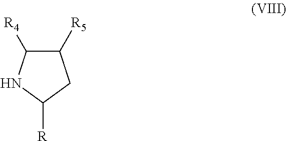

wherein
R is a CONH₂ group, a COOR₂ group or a CN group;
R₂ is a linear or branched C₁₋₁₀ alkyl or an optionally substituted benzyl group;
R₄ and R₅ taken together form a double bond or a three member ring;
in the presence of a condensing agent and a base in a polar aprotic solvent to give the compounds of formula (VI) wherein X is a NR₁ group wherein R₁ have the above reported meanings.

The processes object of the present invention for the preparation of the compounds of formula (VI) are optionally followed by the conversion of the CONH₂ group into CN, when R is a CONH₂ group or by the conversion of the COOR₂ group, wherein R₂ have the above reported meanings, into a CONH₂ and subsequently into CN, when R is a COOR₂ group, according to know techniques in the state of the art.

The processes object of the present invention for the preparation of the compounds of formula (VI) where R₄ and R₅ taken together form a three member ring, optionally comprise the cyclopropanation reaction of the compounds of formula (VI) wherein R₄ and R₅ together form a double bond according to methods known in the art. Preferably, the cyclopropanation reaction is carried out on the compounds of formula (VId) and (VIe).

In steps a) and b1) of the processes object of the present invention, the condensing agent is preferably selected among 2,4,6-tri-n-propyl-2,4,6-trioxo-1,3,5,2,4,6-trioxa-triphosporinane, carbonyldiimidazole, dicyclohexylcarbodiimide. Preferably, 2,4,6-tri-n-propyl-2,4,6-trioxo-1,3,5,2,4,6-trioxa-triphosphorinane is used.

The base is preferably a tertiary amine selected among N,N-diisopropylethylamine, triethylamine, trimethylamine, preferably N,N-diisopropylethylamine. The solvent is preferably selected among acetonitrile, dimethylformamide and dimethylacetamide, acetonitrile being preferred.

In steps b) and a1) of the processes object of the present invention the base is preferably sodium acetate or the acid is preferably acetic acid. The solvent is preferably selected among water, methanol, ethanol or mixtures thereof. A mixture of methanol and water is preferably used.

A preferred embodiment of the process object of the present invention comprises:
a) the condensation reaction between the compound of formula (VII) and a compound of formula (V) in the presence of 2,4,6-tri-n-propyl-2,4,6-trioxo-1,3,5,2,4,6-trioxa-triphosphorinane in acetonitrile, in the presence of N,N-diisopropylethylamine to give the compound of formula (VIa) followed by the conversion of the CONH₂ group into CN group to give the compound of formula (VIb);
b) the reaction of the compound of formula (VIb) with hydroxylamine hydrochloride in the presence of sodium acetate in a mixture of methanol and water to give the corresponding oxime of formula (VIg);

The compound of formula (VI) are new and are intermediates useful for the synthesis of saxagliptin.

The intermediates of formula (VI) can be reduced to give the compounds of formula (X)

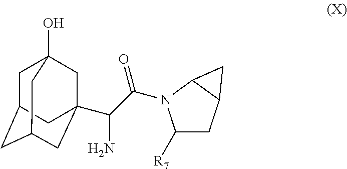

which represent saxagliptin when R₇ is a CN group or represent saxagliptin precursors when R₇ is a CONH₂ group.

Therefore a further object of the present invention is a process for the synthesis of the compounds of formula (X)

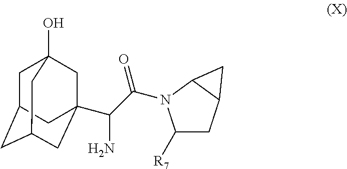

wherein

R₇ is a CONH₂ group or a CN group,

comprising the reduction reaction of a compound of formula (VI-1)

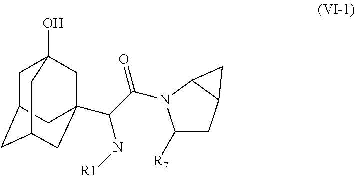

wherein R₇ has the above reported meanings;

R₁ is a hydrogen atom, a hydroxy group, an optionally substituted benzyl or a OR₃ group;

R₃ is a linear or branched C₁₋₁₀ alkyl group, an acetyl group, an optionally substituted benzyl group or a SO₂R₆ group;

R₆ is a linear or branched C₁₋₁₀ alkyl group or an optionally substituted aryl group;

in the presence of hydrogen or hydrogen donors or hydrides, optionally in the presence of a suitable catalyst or in the presence of reducing metals in a protic polar solvent.

Preferably, the hydride is selected among sodium borohydride, lithium aluminiumhydride, Vitride®, diisobutyllithiumaluminiumhydride, sodium triacetoxyborohydride, borane, borane-tetrahydrofuran, triethylsilylhydride, diphenylchlorosilane, zinc borohydride, zinc-pyridine tetrahydroborate complex. Lithium triacetoxyborohydride is preferably used.

Preferably, the reducing metal is selected among sodium, lithium, potassium and magnesium, more preferably sodium. The protic polar solvent is preferably selected among alcohols such as methanol, ethanol, ter-butanol, preferably ter-butanol.

The catalyst is a metal preferably selected among nickel, palladium, platinum, ruthenium, iron, rhodium as such or supported on inert materials or as salts or in the presence of binding agents such as for example amine or phosphinic binding agents, etc. Ni-Raney is preferably used. The solvent is preferably selected among linear or branched C₁-C₄ alcohols such as methanol, ethanol, isopropanol; ethyl acetate, butyl acetate and toluene or mixtures thereof. Methanol is preferably used. Alternatively the compounds of formula (X)

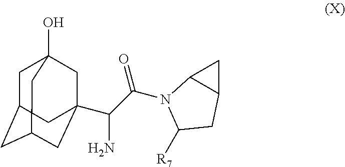

wherein R₇ is a CONH₂ group or a CN group,

can be prepared through a process that is a further object of the present invention comprising a transamination reaction of compounds of formula (VI-2)

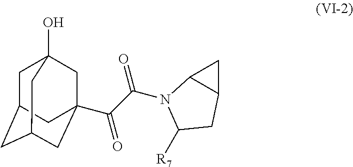

wherein R₇ has the above reported meanings.

The transamination reaction of the compounds of formula (VI-2) can be carried out by enzymatic transamination in the presence of an amino acid dehydrogenase in the presence of a base or an acid to adjust the pH value at about 7; or alternatively by a biomimetic transamination reaction in the presence of an optionally substituted benzyl amine or an amino acid in the presence of a suitable catalyst.

Preferably in the enzymatic transamination reaction the amino acid dehydrogenase is selected among various forms of the phenylalanine dehydrogenase enzyme (PDH) in combination with the formate dehydrogenase (FDH) enzyme in the presence of ammonium formate, dithiothreitol (DTT), nicotinamide adenine dinucleotide (NAD) and using ammonium hydroxide to adjust the pH at about 7.

In the biomimetic transamination reaction the benzyl amine is preferably selected among o-CIPhCH₂NH₂ and o-OHPhCH₂NH₂. The catalyst is preferably selected among sparteine, cinchonine, quinine or proline derivatives, more preferably is a cinchonine derivative of formula (XI)

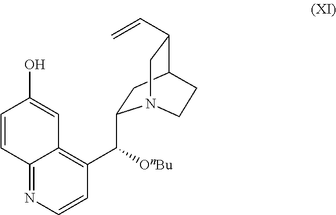

A preferred embodiment of the present invention is the synthesis of saxagliptin comprising the condensation reaction between the compound of formula (VII) and L-cyclopropylprolinamide in the presence of 2,4,6-tri-n-propyl-2,4,6-trioxo-1,3,5,2,4,6-trioxa-triphosphorinane in acetonitrile in the presence of N,N-diisopropylethylamine to give the compound of formula (VIa) followed by the conversion of the CONH₂ group into CN group to give the compound of formula (VIb). The compound of formula (VIb) is reacted with hydroxylamine hydrochloride in the presence of sodium acetate in a mixture of methanol and water, to give the corresponding oxime of formula (VIg) subsequently reduced in the presence of Ni-Raney.

All the terms used in the present application, unless otherwise indicated, are to be understood in their common meaning as known in the art. Other more specific definitions for certain terms, as indicated in this patent application, are underlined later and are constantly applied for the whole description and the claims unless a different definition provides specifically a wider meaning.

For the purposes of the present description and claims which follow, except otherwise indicated, all the numbers that express quantity, quantitative, percentage and so forth, must be considered as modified in all the cases by the term “about”. Moreover, all the ranges include any combination of maximum and minimum points described and include inside them any intermediate range that may or not be specifically enumerated in the present application.

The term “polar solvent” refers to a solvent which is a proton donor, such water; an alcohol, for example methanol, ethanol, propanol, iso-propanol, butanol, tert-butanol; or a polarized solvent such as for example esters, for example ethyl acetate, butyl acetate; nitriles, for example acetonitrile; ethers for example tetrahydrofuran, dioxane; ketones for example acetone, methylbutylketone and the like.

The term “apolar solvent” refers to a solvent which does not behave as a proton donor. Examples include, without limitations, hydrocarbons, such as pentane, hexane, heptane, cyclopentane, cyclohexane; aromatic solvents such as benzene, toluene, o-, m- or p-xilenes; alogenated hydrocarbons such as methylene chloride, chloroform and similar; heterocycles such as tetrahydrofuran, N-methylpyrrolidone; ethers such as diethyl ether, dioxolane etc.

Further information about non polar or polar solvents can be found in organic chemistry books or in specialized monographs, for example, Organic Solvents Physical Properties and Methods of Purification, 4th ed., John A. Riddick, et al., Vol II, in “Techniques of Chemistry Series”, John Wiley & Sons, NY, 1986. Such solvents are known to the person skilled in the art and it is moreover clear to the person skilled in the art that different solvents or mixtures thereof can be selected and preferred, depending on the specific reagents and on the reaction conditions, being their choice influenced, for example, by solubility and reagent reactivity, by preferred temperature ranges.

Although the present invention has been described in its characterizing features, the equivalents and modifications obvious to the skilled in the art are included in the present invention.

The present invention will be now illustrated through some examples without limiting the scope of the invention.

### EXAMPLES

### Example 1

### Synthesis of (1S,3S,5S)-2-(2-((1R,3R,5R,7S)-3-hydroxyadamantan-1-yl)-2-oxoacetyl)-2-azabicyclo[3.1.0]hexan-3-carboxamide—Compound (VIa)

4.00 g of the compound of formula (VII) (0.0178 mol), 36 ml acetonitrile, 2.24 g L-cyclopropylprolinamide (0.0178 mol) and 5.52 g N,N-diisopropylethylamine (0.0427) were charged into a reaction flask. The temperature was brought to −5° C. and 13.55 g of 2,4,6-tri-n-propyl-2,4,6-trioxo-1,3,5,2,4,6-trioxa-triphosphorinane (0.0213 mol) were added. The temperature was brought to 15° C. and the reaction mixture was kept under such conditions for about 90 minutes. At the end of the reaction the solvent was removed by vacuum distillation, 50 ml of water were added and the mixture was kept under stirring for about 90 minutes. The resultant solid was filtered and dried in oven under vacuum at 45° C. to give 4.38 g of compound of formula (VIa).

¹H-NMR (DMSO, 300 MHz): δ 7.41 (s, 1H), 6.97 (s, 1H), 4.61 (m, 1H), 3.19 (m, 1H), 2.18 (s, 2H), 1.85 (m, 2H), 1.76 (m, 4H), 1.65 (m, 2H), 1.55 (m, 6H), 1.20 (m, 1H), 1.08 (m, 1H), 0.67 (m, 1H).

### Example 2

### Synthesis of (1S,3S,5S)-2-(2-((1R,3R,5R,7S)-3-hydroxyadamantan-1-yl)-2-oxoacetyl)-2-azabicyclo[3.1.0]hexan-3-carbonitrile—Compound (VIb)

8.50 g of the compound of formula (VIa) (0.0256 mol), 127 ml ethyl acetate were charged into a reaction flask and the temperature was brought to 0° C. 18.18 g of ethylnicotinate (0.1203 mol) and 15.05 g trifluoroacetic anhydride (0.0717 mol) were added and the reaction mixture was kept under such conditions for about 60 minutes. At the end of the reaction the temperature was brought to about 10-15° C. and 80 ml water and 5 ml benzylamine were added. The organic phase was washed with 45 ml hydrochloric acid 2 N solution and 60 ml of a 25% potassium carbonate solution in water and 17 ml of methanol were then added. The temperature was brought to 40° C. and the reaction mixture was kept under such conditions for 60 minutes. At the end of the reaction the reaction mixture was washed with a 2N hydrochloric acid solution (2×40 ml) and with water (1×20 ml) and the pH was adjusted to about 7 by adding a 1M sodium hydroxide solution. The collected organic phases were concentrated to residue by distillation under vacuum to give 6.20 g of the compound of formula (VIb).

¹H-NMR (DMSO, 300 MHz): δ 5.22 (d, 1H), 4.71 (s, 1H), 3.37 (m, 1H), 2.62 (m, 1H), 2.28 (m, 1H), 2.20 (s, 2H), 1.88 (m, 1H), 1.75 (m, 6H), 1.56 (m, 6H), 0.96 (m, 1H), 0.75 (m, 1H).

### Example 3

### Synthesis of (1S,3S,5S)-2-(2-((1R,3R,5R,7S)-3-hydroxyadamantan-1-yl)-2-(hydroxyimino)acetyl)-2-azabicyclo[3.1.0]hexan-3-carboxamide—Compound (VIf)

4.00 g of the compound of formula (VIa) (0.0120 mol), 20 ml methanol, 1.97 g sodium acetate (0.0240 mol), 8 ml water and 1.67 g hydroxylamine hydrochloride (0.0240 mol) were charged into a reaction flask. The temperature was brought to 40° C. and the reaction mixture was kept under such conditions for about 23 hours. At the end of the reaction the solvent was removed by distillation under vacuum, 40 ml of water were added and the temperature was slowly brought to about 15° C. The resultant solid was filtered, cool washed with water (2×6 ml) and dried in oven under vacuum at 45° C. to give 6 g of the compound of formula (VIf).

¹H-NMR (DMSO, 300 MHz): δ 10.94 (s, 1H), 7.30 (s, 1H), 6.88 (s, 1H), 4.63 (m, 1H), 4.45 (s, 1H), 3.17 (m, 1H), 2.10 (s, 2H), 1.88 (m, 2H), 1.75 (m, 7H), 1.56 (m, 6H), 0.88 (m, 1H), 0.67 (m, 1H).

### Example 4

### Synthesis of (1S,3S,5S)-2-(2-((1R,3R,5R,7S)-3-hydroxyadamantan-1-yl)-2-(hydroxyimino)acetyl)-2-azabicyclo[3.1.0]hexan-3-carbonitrile-Compound (VIf)

0.400 g of the compound of formula (VIb) (0.00127 mol), 2.5 ml methanol, 0.8 ml water and 0.176 g hydroxylamine hydrochloride (0.00254 mol) were charged into a reaction flask, the temperature was brought to 40° C. and the reaction mixture was kept under such conditions overnight. At the end of the reaction a saturated sodium chloride solution was added and the mixture extracted with methylene chloride (3×4 ml). The collected organic phases were concentrated to residue by distillation under vacuum to give 0.150 g of the compound of formula (VIg).

¹H-NMR (DMSO, 300 MHz): δ 10.94 (s, 1H), 4.63 (m, 1H), 4.45 (s, 1H), 3.17 (m, 1H), 2.10 (s, 2H), 1.88 (m, 2H), 1.75 (m, 7H), 1.56 (m, 6H), 0.88 (m, 1H), 0.67 (m, 1H).

### Example 5

### Synthesis of (1S,3S,5S)-2-((S)-2-amino-2-((1R,3R,5R,7S)-3-hydroxyadamantan-1-yl)acetyl)-2-azabicyclo[3.1.0]hexan-3-carboxamide—Compound (X) wherein R═CONH₂

0.300 g of the compound of formula (VIg) (0.0008 mol), 2 ml methanol, 1 ml Ni-Raney were charged into a reaction flask and the reaction mixture was put under hydrogen atmosphere and kept under such conditions overnight. At the end of the reaction the mixture reaction was separated by column chromatography and the solvent was removed by vacuum distillation to give 0.180 g of the compound of formula (X) wherein R═CON H₂.

¹H-NMR (CDCl₃, 300 MHz): δ 8.81 (s, 2H), 7.25 (s, 1H), 6.81 (s, 1H), 4.29 (m, 1H), 4.20 (s, 1H), 3.65 (m, 1H), 2.33 (m, 2H), 2.05 (s, 2H), 1.85 (m, 1H), 1.59 (m, 12H), 1.10 (m, 1H), 0.67 (m, 1H).

### Example 6

### Synthesis of Saxagliptin (Biomimetic Transamination)

100 mg of the compound of formula (VIb) (0.00032 mol), 2 ml methanol, 0.5 ml water, 77 mg pyridoxamine dihydrochloride (0.00032 mol), 44 mg potassium carbonate (0.00032 mol) and 10 mg cinchonidine (0.000032 mol) were charged into a reaction flask. The temperature was brought to about 50° C. and the reaction mixture was kept under such conditions for about 48 hours. At the end of the reaction, the resultant solid was filtered and washed with 0.5 ml methanol, purified by column chromatography and dried in oven under vacuum at 40° C. to give 60 mg of saxagliptin.

1. Compounds of formula (VI) 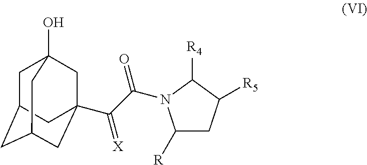
wherein
X is an oxygen atom or a NR₁ group;
R is a CONH₂ group, a COOR₂ group or a CN group;
R₁ is a hydrogen atom, a hydroxy group, an OR₃ group, or an optionally substituted benzyl group;
R₂ is a linear or branched C₁₋₁₀ alkyl group, an optionally substituted benzyl group;
R₃ is a linear or branched C₁₋₁₀ alkyl group, an acetyl group, an optionally substituted benzyl group, a SO₂R₆ group;
R₄ and R₅ taken together form a double bond or a three member ring;
R₆ is a linear or branched C₁₋₁₀ alkyl group, or an optionally substituted aryl group; and their stereoisomers.

2. A compound according to claim 1, selected from the group consisting of formula 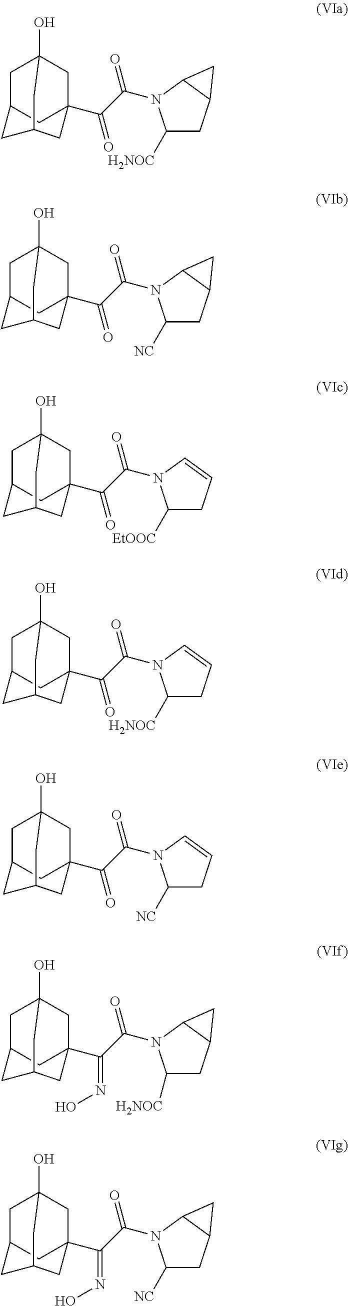 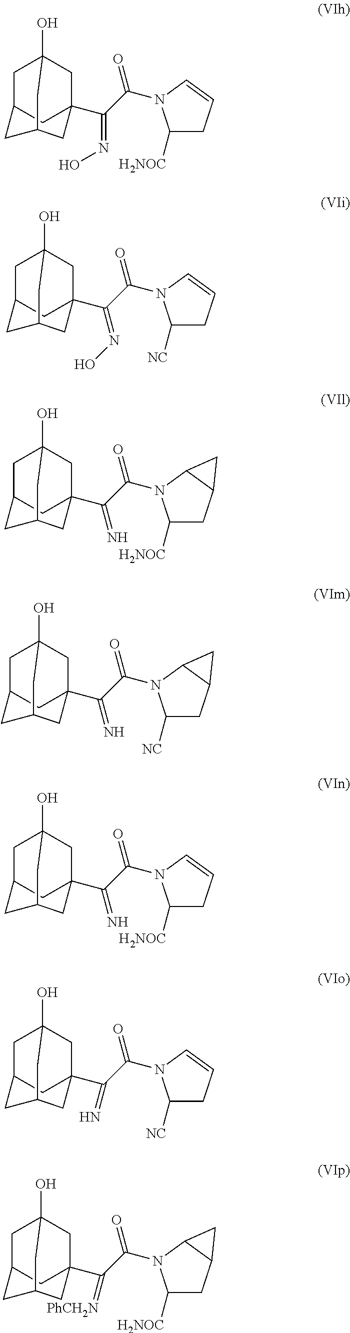 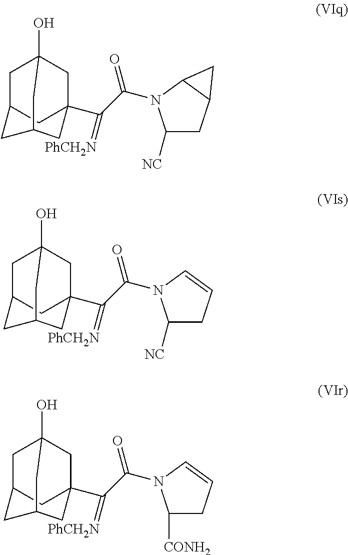
and stereoisomers thereof.

3. A process for the preparation of compounds of formula (VI), 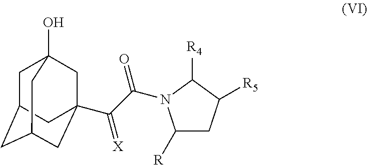
wherein
X is an oxygen atom or a NR₁ group;
R is a CONH₂ group, a COOR₂ group or a CN group;
R₁ is a hydrogen atom, a hydroxy group, an OR₃ group, or an optionally substituted benzyl group;
R₂ is a linear or branched C₁₋₁₀ alkyl group, an optionally substituted benzyl group;
R₃ is a linear or branched C₁₋₁₀ alkyl group, an acetyl group, an optionally substituted benzyl group, a SO₂R₆ group;
R₄ and R₅ taken together form a double bond or a three member ring;
R₆ is a linear or branched C₁₋₁₀ alkyl group, or an optionally substituted aryl group;
comprising:
a) reacting an adamantane derivative of formula (VII) 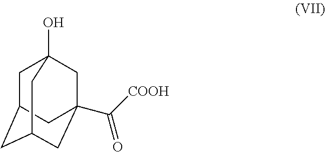 [SMILES: O=C(O)C(=O)C12CC3CC(CC(O)(C3)C1)C2]
with a compound of formula (VIII) 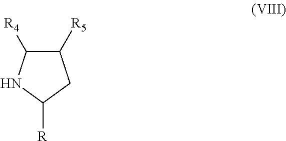
wherein R, R₄ and R₅ have the above reported meanings;
in the presence of a condensing agent and optionally in the presence of a base, in an aprotic solvent to give compounds of formula (VI) wherein X represents an oxygen atom; and optionally
b) reacting the compound formula (VI) obtained in step a) with NH₂R₁, in the presence of a base or an acid, in a polar solvent to give compounds of formula (VI) wherein X represents a N—R₁ group, wherein R₁ has the above reported meanings.

4. A process for the preparation of compounds of formula (VI), 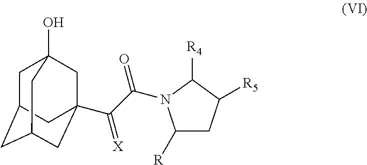
wherein
X is an oxygen atom or a NR₁ group;
R is a CONH₂ group, a COOR₂ group or a CN group;
R₁ is a hydrogen atom, a hydroxy group, an OR₃ group, or an optionally substituted benzyl group;
R₂ is a linear or branched C₁₋₁₀ alkyl group, an optionally substituted benzyl group;
R₃ is a linear or branched C₁₋₁₀ alkyl group, an acetyl group, an optionally substituted benzyl group, a SO₂R₆ group;
R₄ and R₅ taken together form a double bond or a three member ring;
R₆ is a linear or branched C₁₋₁₀ alkyl group, or an optionally substituted aryl group;
comprising:
a1) reacting an adamantane intermediate of formula (VII) 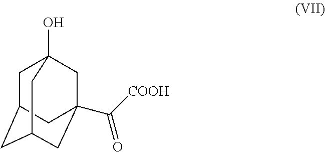 [SMILES: O=C(O)C(=O)C12CC3CC(CC(O)(C3)C1)C2]
with NH₂R₁ wherein R₁ has the above reported meanings, in the presence of a base or an acid, in a polar solvent to give compounds of formula (IX) 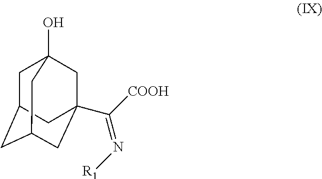
wherein R₁ has the above reported meanings; and
b1) reacting the compound of formula (IX) obtained in step a1) and a compound of formula (VIII) 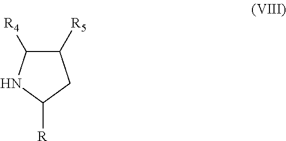
wherein R, R₄ and R₅ have the above reported meanings;
in the presence of a condensing agent and a base, in a polar aprotic solvent to give compounds of formula (VI) wherein X and R₁ have the above reported meanings.

5. A process according to claim 3 comprising the conversion of the CONH₂ group into CN group, when R is a CONH₂ group, or the conversion of the COOR₂ group, wherein R₂ is a linear or branched C₁₋₁₀ alkyl group, an optionally substituted benzyl group, into a CONH₂ group and subsequently into CN group, when R is a COOR₂ group wherein R₂ have the above reported meanings.

6. A process according to claim 3 comprising the cyclopropanation reaction of the compounds of formula (VI) wherein R₄ and R₅ taken together form a double bond.

7. A process for the preparation of compounds of formula (X) 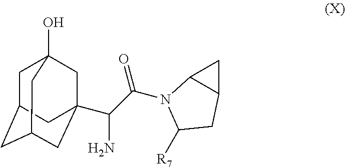
wherein R₇ is a CONH₂ group or a CN group,
comprising reacting a compound of formula (VI-1) 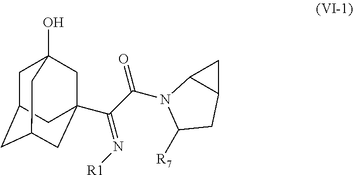
wherein
R₇ has the above reported meanings;
R₁ is a hydrogen atom, a hydroxy group, an OR₃ group, or an optionally substituted benzyl group;
R₃ is a linear or branched C₁₋₁₀ alkyl group, an acetyl group, a optionally substituted benzyl group, a SO₂R₆ group;
R₆ is a linear or branched C₁₋₁₀ alkyl group, or an optionally substituted aryl group;
with hydrogen, or hydrogen donor compounds, or hydrides, optionally in the presence of a suitable catalyst; or with reducing metals in the presence of a polar protic solvent.

8. A process for the preparation of compounds of formula (X) 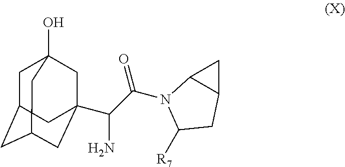
wherein R₇ represents a CONH₂ group or a CN group,
comprising transaminating of compounds of formula (VI-2) 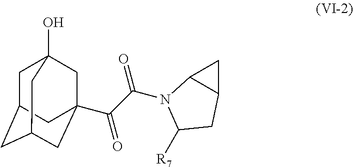
wherein R₇ has the above reported meanings.

9. A process according to claim 3 comprising the condensation reaction of the compound of formula (VII) and L-cyclopropylprolinamide in the presence of 2,4,6-tri-n-propyl-2,4,6-trioxo-1,3,5,2,4,6-trioxa-triphosphorinane, in acetonitrile, in the presence of N,N-diisopropylethylamine to give the compound of formula (VIa) followed by the conversion of the CONH₂ group into CN group to give the compound of formula (VIb) followed by the reaction of the compound of formula (VIb) with hydroxylamine hydrochloride in the presence of sodium acetate, in a mixture of methanol and water, to give the corresponding oxime of formula (VIg), subsequently reduced in the presence of Ni-Raney.

10. A process according to claim 3 for the preparation of Saxagliptin.
